(19) United States
(12) Patent Application Publication
(10) Pub. No.: US 2010/0233746 A1
(43) Pub. Date: Sep. 16, 2010

(54) Inflammation and Oxidative Stress Level Assay

(76) Inventors: Denise Sonntag, Innsbruck (AT); Therese Koal, Innsbruck (AT); Steven Lewis Ramsay, Viewbank (AU); Sascha Dammeier, Goetzens (AT); Klaus Michael Weinberger, Mieming (AT); Ines Unterwurzacher, Innsbruck (AT)

Correspondence Address: THORPE NORTH & WESTERN, LLP., P.O. Box 1219, SANDY, UT, 84091-1219, US

(21) Appl. No.: 12/602,471

(22) PCT Filed: May 30, 2008
(86) PCT No.: PCT/EP08/04323

§ 371 Date: Apr. 22, 2010

Related U.S. Application Data
(60) Provisional application No. 60/924,820, filed on May 31, 2007.

(51) Int. Cl. C12Q 1/26; C12Q 1/00

(52) U.S. Cl. 435/25; 435/4

(57) ABSTRACT

The present invention relates to a method for determining the systemic metabolic status of an organism in relation to inflammation and oxidative stress using a biological sample (Inflammation and Oxidative Stress Level Assay). This comprises detection and quantification of one or more derivatives of arachidonic acid (eicosanoids), linoleic acid and/or docosahexaenoic acid, preferably together with one or more oxidative stress parameters and/or with one or more analytes from other metabolite classes in parallel, as well as a kit adapted for carrying out such a method. Moreover, the invention relates to the biomarkers as employed in the method.

### TECHNICAL FIELD

This invention relates to a method for determining the systemic metabolic status of an organism in relation to inflammation and oxidative stress using a biological sample (Inflammation and Oxidative Stress Level Assay). This comprises detection and quantification of one or more derivatives of arachidonic acid (eicosanoids), one or more derivatives of linoleic acid and/or one or more derivatives of docosahexaenoic acid, preferably together with one or more oxidative stress parameters and/or with one or more analytes from other metabolite classes in parallel, as well as a kit adapted for carrying out such a method. Moreover, the invention relates to the biomarkers as employed in the method.

### BACKGROUND OF THE INVENTION

Inflammation is a local response to cellular injury that is marked by capillary dilatation, leukocyte infiltration, redness, heat, pain, swelling, and often loss of function and that serves as a mechanism initiating the elimination of noxious agents and damaged tissue [Webster's Medical Desk Dictionary. Merrian-Webster. 1986]. When an inflammatory stimulus is sufficiently strong, a systemic inflammatory response syndrome (SIRS) will develop.

Prostaglandins are the key mediators of inflammation, pain, fever and anaphylactic reactions. A wide variety of other biological processes is directly or indirectly influenced by the action of prostanoids: hemostasis, platelet aggregation, kidney and gastric function, female reproduction, angiogenesis, immunological functions, development and cancer [Williams, C. S. et al., Oncogene 1999, 18, 7908-16; Rocca, B. et al., J. Clin Invest 1999, 103, 1469-77; Howe, L. R. Breast Cancer Res. 2007, 9, 210].

Methods for the measurement of inflammation have been described, for example, in WO 2003/014699, WO 2006/124714, and WO 2004/025303.

Oxidative stress has been defined as “a disturbance in the pro-oxidant/antioxidant balance in favor of the former, leading to possible [tissue] damage” [Sies, H., Oxidative Stress. Oxidants and Antioxidants. 1991, New York: Elsevier. 507]. It has been implicated as a key common pathway for cellular dysfunction and death and a potential therapeutic target in a broad spectrum of human medical conditions including cancer, diabetes, obstructive lung disease, inflammatory bowel disease, cardiac ischemia, glomerulonephritis, macular degeneration and various neurodegenerative disorders [Halliwell, B. and J. M. C. Gutteridge, Free Radicals in Biology and Medicine. 3 ed. 1999, Oxford: Oxford University Press Inc. 736]. Oxidative stress measurement devices and methods have been described, for example, in WO 2005/052575, WO 2006/127695, JP 2003083977, U.S. Pat. No. 5,891,622, U.S. Pat. No. 6,620,800, WO 2003/016527, U.S. Pat. No. 6,096,556, WO 1998/10295, WO 2006/90228, WO 2002/04029, WO 1999/63341, EP 0 845 732, WO 2007/041868, WO 2007/083632.

Inflammation and oxidative stress are closely related. Phagocytes, i.e. macrophages and neutrophils, are activated in inflammation. To combat pathogens, they produce reactive oxygen species, which are key mediators of oxidative damage. They are toxic for microorganisms but can also lead to tissue injury.

Some of the end products of the cell/tissue damage, such as 3-nitrotyrosine for the nitration of proteins, 4-hydroxy-2′-nonenal and malondialdehyde for the lipid peroxidation, or 8-hydroxyguanosine for nucleic acid damage, are already known, however, the detection processes are complicated and not sufficiently sensitive in order to detect gradual changes of the oxidative stress indicating, for example, beneficial therapy effects.

Only minor efforts have been made to combine oxidative stress measurement with the determination of parameters that are involved in inflammation. For example, WO 02/100293 describes a diagnostic and prognostic method for evaluating ocular inflammation and oxidative stress and the treatment of the same, whereas WO 02/090977 describes a method to test substances for inflammatory or oxidant properties.

Recent developments have focused on the detection of a specific class of oxidative stress parameters, namely the prostanoids and isoprostanes (Masoodi, M. et al, Rap Comm Mass Spec 2006, 20, 3023; Taylor, A. W. et al, Analyt. Biochem. 2006, 350, 41). However, these methods necessarily use solid phase extraction or liquid-liquid extraction procedures, which require a minimum sample volume of 500 often a derivatization process with complex workup methods, followed by evaporation and resolvation steps. Moreover, these processes have been described only for the analysis of prostanoids and isoprostanes using HPLC or LC tandem mass spectrometry procedures.

In view of the above problems existing in the prior art, it is an object underlying the present invention to provide for an improved method for determining the systemic metabolic status in relation to inflammation and oxidative stress in a biological sample which method is highly sensitive and allows for the detection of only slight changes in the systemic metabolic status.

Moreover, it is an object to provide a kit for carrying out such a method.

### SUMMARY OF THE INVENTION

The present invention provides a method for the concurrent determination of inflammation and oxidative stress level parameters in a biological sample which comprises detection of one or more derivatives of arachidonic acid (eicosanoids), linoleic acid and/or docosahexaenoic acid, preferably together with one or more oxidative stress parameters and analytes from other chemical classes, respectively, in parallel, and a kit adapted for carrying out this method. Moreover, the derivatives of arachidonic acid (eicosanoids), linoleic acid and docosahexaenoic acid, as well the other oxidative stress parameters and analytes from other chemical classes are detected by measuring metabolite concentrations employing a quantitative analytical method such as chromatography, spectroscopy, and mass spectrometry. Particularly preferable is the use of the methods and devices as described in WO 2007/003344 and WO 2007/003343, whose applications are both incorporated herein by reference.

### DESCRIPTION OF THE PREFERRED EMBODIMENTS

Bioactive lipids of prostanoid structure and hydroxylated fatty acid derivatives play a central role in the metabolism of higher organisms. Prostaglandins are key mediators of inflammation, pain, fever and anaphylactic reactions, thromboxanes mediate vasoconstriction, and prostacyclins are active in the resolution phase of inflammation and in cardioprotection. A wide variety of other biological processes is directly or indirectly influenced by the action of prostanoids: hemostasis, platelet aggregation, kidney and gastric function, female reproduction, angiogenesis, immunological functions, development and cancer [Williams, C. S. et al., Oncogene 1999, 18, 7908-16; Rocca, B. et al., J. Clin Invest 1999, 103, 1469-77; Howe, L. R. Breast Cancer Res. 2007, 9, 210].

This diversified functionality makes prostanoids valuable indicators of the overall biological condition of higher organisms. As small concentration changes exert pronounced effects, an imbalance in prostanoid metabolites indicates acute reactions, e.g. local or systemic inflammation, as well as chronic disturbances of biological processes. For a correct assessment of the actual bodily condition, further metabolic parameters must be considered.

Oxidative stress is mainly caused by reactive oxygen species (ROS), which are constantly generated by mitochondrial aerobic respiration, phagooytosis of bacteria or virus-containing cells, and peroxisomal-mediated degradation of fatty acids. [Ames, B. N. et al., Proc. Natl. Acad. Sci. U.S.A 1993, 90, 7915-22]. Increased ROS production occurs in inflammation, during radiation or during metabolism of hormones, drugs, and environmental toxins.

ROS can easily react with lipids forming lipid hydroperoxides of different origin. The ROS-mediated oxidation of esterified linoleic acid-containing lipids and free linoleic acid results in the formation of hydroperoxyoctadecadienoic acid (HPODE) isomers; they are subsequently reduced to the corresponding hydroxyoctadecadienoic acids (HODEs). Oxidation of arachidonic acid-containing lipids and free arachidonic by ROS, on the other hand, leads to the formation of a complex mixture of hydroperoxyeicosatetraenoic acids (HPETEs) that are reduced to hydroxyeicosatetraenoic acids (HETEs) [Blair, I. A., J. Biol. Chem. 2008]. Lipid hydroperoxides can also be formed by lipoxygenases (LOXs) [Ames, B. N. et al., Proc. Natl Acad. Sci. U.S.A 1993, 90, 7915-22] and cyclooxygenases (COXs) [Porter, N. A. et al., Lipids 1995, 30, 277-90] acting on polyunsaturated fatty acids (PUFAs).

Normal metabolic processes generate potentially hazardous reactive oxygen species that lead to oxidative damage and inflammation, while both interconnected processes have in turn a general and pronounced impact on metabolic reactions. This process increases with age. Oxidative stress and inflammation have been implicated in many diseases, e.g. atherosclerosis, hypertension, asthma, COPD, acute lung injury, heart failure, kidney and hepatic diseases.

As for kidney disease, for example, both increased oxidative stress and increased acute phase inflammation, considered as nontraditional risk factors, are postulated as to be important contributors to uremic cardiovascular risk [Himmelfarb, J., Seminars in Dialysis 2008, 17, 449-454(6)]. Oxidative cellular damage occurs frequently in livers with alcoholic and non-alcoholic fatty liver disease, showing strong correlation of 8-hydroxydeoxyguanosine and 4-hydroxy-2′-nonenal indices with necro-inflammation [Seki, S. et al., Histopathology 2003, 42, 365-71; Seki, S. et al. Hepatol. Res. 2005, 33, 132-34].

The concurrent assessment of inflammation- and oxidative stress-related parameters as well as the determination of the overall metabolic status of the organism according to the invention is highly beneficial in respect to diagnosis, treatment, and prognosis of diseases. Ideally, a defined and combined set of biomarkers as obtained according to the invention that cover inflammation, oxidative stress and metabolic aspects of a disease serves as a valuable diagnostic and prognostic tool in health care.

According to the method for determining the systemic status of inflammation, oxidative stress and metabolic disturbances in a biological sample of the present invention one or more derivatives of arachidonic acid (eicosanoids), of linoleic acid and/or of docosahexaenoic acid are detected (hereinafter referred to as the first group of compounds). Preferably these one or more derivatives of arachidonic acid, linoleic acid and/or docosahexaenoic acid are detected in parallel from the same sample.

The derivatives of arachidonic acid are preferably selected from the group consisting of arachidonic acid and its metabolites, such as cyclooxygenase-, lipoxygenase- and cytochrome P450-derived prostanoids, hydroxy-, hydroperoxy- and epoxylated acids and non-enzymatic peroxidation products like isoprostanes.

The derivatives of linoleic acid are preferably selected from the group consisting of linoleic acid and its metabolites, such as lipoxygenase- and cytochrome P450-derived oxidation products, and non-enzymatic peroxidation products.

The derivatives of docosahexaenoic acid preferably are selected from the group consisting of docosahexaenoic acid and its metabolites, such as lipoxygenase- and cytochrome P450-derived docosanoids and non-enzymatic peroxidation products like isoprostanes.

Furthermore, it is preferable according to the present invention to detect the compounds of the first group, i.e. the one or more derivatives of arachidonic acid, linoleic acid and/or docosahexaenoic acid, together with one or more parameters of inflammation and/or oxidative stress from other chemical classes in parallel (hereinafter referred to as the second group of compounds).

These parameters from other chemical classes are, for example, selected from the group consisting of products of lipid oxidation and/or peroxidation, tyrosine derivatives like NO₂-, Br-, Cl-tyrosine, methionine sulfoxide, ketone bodies, 8-oxo-guanidine and 8-OH guanosine, biopterins, pro-vitamins, vitamins, antioxidants, glutathione, ophthalmate, oxidized cholesterols and sterols.

Additionally, it is particularly preferable according to the present invention to detect the one or more derivatives of arachidonic acid, linoleic acid and/or docosahexaenoic acid (first group) and the one or more parameters of inflammation and/or oxidative stress (second group) together with one or more analytes from other metabolite classes in parallel (hereinafter referred to as the third group of compounds). These analytes from other metabolite classes are, for example, selected from the group consisting of α-ketoglutarate, succinate, CoQ₁₀, methionine, sphingolipids, such as ceramide-1-phosphate, sphingosine-1-phosphate, sphingomyelins and hydroxylated sphingomyelins.

Even if it is in principle possible according to the present invention to carry out the method based on any combination of the above compounds (metabolites) of the three groups, the following combinations 1)-6) as shown below are particularly preferred.
1) HETEs/HODEs+methionine sulfoxide+methionine
2) arachidonic acid+ceramide-1-phosphate
3) arachidonic acid+oxidised cholesterols/sterols+sphingomyelins
4) prostaglandins+sphingosine-1-phosphate
5) HETEs/HODEs+8-oxo-guanidine/8-OH-guanosine
6) prostaglandins+NO2-tyrosine

The detection is carried out by measuring one or more metabolite concentrations preferably using the methods and devices as described in WO 2007/003344 and WO 2007/003343 which applications are both incorporated herein by reference. By using these methods, wherein the inserts in the microtiter plate already contain the internal standards, it is possible to avoid commonly used time consuming derivatization processes with complex work up methods as well as additional solid phase extractions or liquid-liquid extraction procedures. Consequently, the method according to the present invention is less time consuming and can be carried out in smaller sample volumes. In particular, it is possible according to the present invention to carry out the detection in a sample having a low volume within a range of from 5 μl to 100 μl and more preferably from 10 μl to 50 μl. Quite in contrast, the prior art processes require a volume of at least 500 μl. These low sample volumes used according to the present invention render the method also an ideal application for small sample volumes, e.g. samples from small animals or studies on newborns. Apart there from, the limit of detection is almost identical with the limits of detection of the prior art, even though the sample volume is significantly decreased according to the present invention.

The biological sample may be obtained from a mammal, preferably from a mouse, a rat, a guinea pig, a dog, a mini-pig, a primate or a human. Thus, the method according to the invention is an in vitro method.

The detection according to the present invention is based on a quantitative analytical method commonly used and known in the prior art, such as chromatography, spectroscopy, and mass spectrometry. Particularly preferable is mass spectrometry, while the specific technique is not particularly limited. Any mass spectrometry may be used according to the present invention comprising usual mass spectrometry techniques, which combine e.g. atmospheric pressure ionization modi or MALDI with single or triple quadrupol-, ion trap-, TOF or TOF-TOF-detection systems.

The systemic metabolic status may be indicative for various kinds of diseases. Examples of diseases which may be relevant according to the present invention are various cancer types, inflammatory diseases such as chronic airway inflammation or atherosclerosis, and metabolic disorders like diabetes. Furthermore, obstructive lung disease, inflammatory bowel disease, cardiac ischemia, glomerulonephritis, macular degeneration and various neurodegenerative disorders may be mentioned. The method of the invention is also useful in detecting the gradual change of oxidative stress e.g. due to therapeutic effects.

Moreover the invention is also directed to a kit adapted for carrying out the method wherein the kit comprises a device which device contains one or more wells and one or more inserts impregnated with at least one internal standard. Such a device is in detail described in WO 2007/003344 and WO 2007/003343 as mentioned above.

Additionally the invention is also directed to the biomarker for determining a systemic metabolic status in relation to inflammation and oxidative stress in a biological sample itself.

The present invention will become more apparent in view of the following examples specifying particularly preferred embodiments.

### EXAMPLES

### Introduction

Free fatty acid metabolites, such as arachidonic acid and its plethora of downstream metabolites, all play important roles in many physiological and pathological processes, including development of different diseases such as various cancer types, diabetes, cardiovascular disease and chronic airway inflammations. In the following experimental setting, it was the focus to determine prostanoids, hydroxy-, hydroperoxy- and epoxylated acids and non-enzymatic peroxidation products like isoprostanes, which derived from cyclooxygenase, lipoxygenase and cytochrome P450 enzyme activity in various biological sample types.

As described in this invention, a rapid method was performed to extract free fatty acid metabolites from 20 μL of plasma and other biological matrices with subsequent analysis by HPLC-MS/MS. The low sample volume used in this method makes it also an ideal application for use in small animal studies.

The analytes that have been quantitatively determined are summarized in table 1.

TABLE 1
List of Analytes

| | |
|---|---|
| Prostaglandins | PGD₂, PGE₂, PGF_{2α}, 6-keto PGF_{1α} |
| Isoprostane | 8-iso PGF_{2α} |
| Leukotrienes | LTB₄, LTD₄ |
| Thromboxane | TXB₂ |
| Hydroxyeicosatetraenoic acids | 12(S)-HETE, 15(S)-HETE |
| Epoxy-, hydroperoxy acids | 5(S)-HpETE, 15(S)-HpETE, |
| | 14(15)-EpETE |
| Hydroxyoctadecadienoic acids | (+−)9-HODE, 13(S)-HODE |
| Fatty acids | Arachidonic Acid, Docosahexaenoic Acid |

### Methods:

To determine free fatty acid metabolites in various biological materials, a small amount of sample (20 μL) was applied onto a filter spot containing stable isotopes for the various metabolites in a microtiter plate and extracted in aqueous methanol without further derivatization as described in FIG. 1. Separation of metabolites was done on a RP-HPLC (Zorbax Eclipse C18, 3.0×100 mm, 3.5 μm) column after injection of 20 μL extract using an Agilent 1100 system (Agilent Technologies) with an HTC PAL autosampler (CTC Analytics). Mobile phase compositions were A: H2O with 0.05% (v/v) formic acid and B: acetonitrile with 0.05% (v/v) formic acid. Flow rate was constant at 500 μL/min, metabolites were separated by gradient elution. Detection was done by MRM transitions in negative detection mode using an API4000Qtrap® equipped with an ESI source (Applied Biosystems). Quantification of metabolites was performed with Analyst v.1.4.2 quantitation. Representative chromatograms of a standard mixture are shown in FIGS. 2a and 2b.

### Sample Preparation:

Plasma preparation was performed in EDTA-coated vials containing 0.001% BHT (butylated hydroxytoluene). Homogenates of brain, liver and prostate tissue were prepared in PBS-buffer.

### Method Validation:

The method validation was performed with human plasma. Following internal standards were used for quantification: 12(S)-HETE-d₈, PGE₂-d₄, PGD2-d₄, TXB₂-d₄, PGF₂_{_{α}}-d₄, 6-keto PGF₁_{_{α}}-d₄ and DHA-d₅. Linearity of the assay was determined with a 6-point calibration curve, applying a 1/x weighting factor to the data. Lower limit of quantification (LLOQ) and limit of detection (LOD) were determined by spiking plasma samples with external standard solution and diluting with PBS to the expected quantification limit. Linear ranges of analytes, correlation coefficients and values for LLOQ and LOD are listed in table 2.

TABLE 2
Overview of compounds, correlation coefficients,
linear ranges, LLOQ and LOD

| | Correlation | Linear range | LLOQ | LOD |
|---|---|---|---|---|
| Compound | coefficient | [nmol/L] | [nmol/L] | [nmol/L] |
| (±)9-HODE | 0.9985 | 50-5000 | 50 | 27 |
| 13(S)-HODE | 0.9997 | 1-100 | 0.4 | 0.05 |
| 12(S)-HETE | 0.9988 | 5-500 | 2 | 0.6 |
| 15(S)-HETE | 0.9992 | 5-500 | 2 | 1.3 |
| 5(S)-HpETE | 0.9987 | 48-4800 | 143 | 35 |
| 15(S)-HpETE | 0.9992 | 36-3600 | 14 | 8 |
| 14(15)-EpETE | 0.9976 | 50-5000 | 10 | 3 |
| LTB₄ | 0.9992 | 5-500 | 10 | 0.26 |
| LTD₄ | 0.9991 | 12-1200 | 7 | 5.5 |
| PGD₂ | 0.9994 | 5-500 | 1 | 1.0 |
| PGE₂ | 0.9991 | 5-500 | 0.75 | 0.65 |
| PGF_{2α} | 0.9994 | 5-500 | 4 | 1.2 |
| 8-iso PGF_{2α} | 0.9995 | 5-500 | 4 | 3.6 |
| 6-keto PGF_{1α} | 0.9981 | 50-5000 | 5 | 3.8 |
| TXB₂ | 0.9998 | 5-500 | 3 | 1.6 |
| AA | 0.9976 | 388-38800 | 233 | 188 |
| DHA | 0.9990 | 200-20000 | 20 | 17 |

Intraday and interday reproducibilities were determined by replicate injections (n=5) of plasma spiked at three concentrations over six consecutive days. Assay accuracies were calculated by comparing mean concentrations to the true values of the analytes (n=5). Average coefficients of variance (CV) for intraday and interday precision and accuracy of each compound are shown in table 3.

TABLE 3
coefficients of variance (CV) of intraday and interday reproducibility,
and accuracies of fatty acid metabolites in spiked plasma samples

| | Intraday | | | Interday | | | | | |
|---|---|---|---|---|---|---|---|---|---|
| | reproducibility | | | reproducibility | | | Accuracy [%] | | |
| | CV [%], n = 5 | | | CV [%], n = 6 | | | n = 5 | | |
| Compound | low | medium | high | low | medium | high | low | medium | high |
| (±)9-HODE | 5.4 | 8.8 | 7.1 | 12.6 | 11.0 | 8.0 | 78.2 | 88.4 | 82.3 |
| 13(S)-HODE | 6.0 | 7.8 | 5.9 | 9.8 | 10.4 | 8.8 | 94.9 | 91.5 | 84.4 |
| 12(S)-HETE | 5.3 | 8.7 | 7.3 | 7.5 | 10.2 | 9.0 | 103.5 | 112.5 | 100.6 |
| 15(S)-HETE | 14.3 | 15.1 | 7.8 | 31.8 | 44.5 | 18.9 | 184.6 | 232.7 | 191.4 |
| 5(S)-HpETE | 35.1 | 27.6 | 23.0 | 55.6 | 45.8 | 45.7 | 45.9 | 68.6 | 67.1 |
| 15(S)-HpETE | 14.4 | 13.6 | 15.9 | 39.8 | 36.1 | 32.2 | 90.8 | 81.8 | 77.2 |
| 14(15)-EpETE | 6.4 | 7.1 | 8.2 | 9.9 | 11.9 | 12.0 | 71.1 | 126.0 | 116.4 |
| LTB₄ | 10.0 | 7.6 | 7.8 | 10.1 | 11.6 | 9.7 | 61.4 | 98.1 | 86.6 |
| LTD₄ | 6.0 | 7.4 | 7.2 | 7.8 | 8.9 | 7.2 | 79.8 | 139.8 | 125.1 |
| PGD₂ | 3.9 | 4.1 | 6.2 | 5.3 | 5.7 | 6.5 | 109.1 | 124.8 | 113.0 |
| PGE₂ | 2.4 | 4.7 | 5.6 | 5.4 | 5.7 | 6.5 | 113.0 | 116.5 | 111.3 |
| PGF₂_{_{α}} | 9.0 | 7.0 | 8.1 | 9.4 | 10.5 | 9.7 | 134.6 | 121.9 | 107.1 |
| 8-iso PGF₂_{_{α}} | 7.5 | 9.6 | 5.8 | 12.6 | 11.6 | 10.6 | 156.3 | 114.0 | 100.8 |
| 6-keto PGF₁_{_{α}} | 3.7 | 4.7 | 5.7 | 7.3 | 7.3 | 7.7 | 71.4 | 121.6 | 107.8 |
| TXB₂ | 7.1 | 5.6 | 4.6 | 10.4 | 9.0 | 6.4 | 89.2 | 117.6 | 96.8 |
| AA | 12.8 | 9.8 | 11.7 | 13.6 | 13.0 | 16.3 | 32.0 | 75.3 | 85.1 |
| DHA | 4.3 | 6.0 | 5.1 | 9.6 | 8.1 | 9.3 | 86.0 | 103.5 | 97.3 |

The validation procedure exhibited following values:
Lower limits of quantification were all 0.4-50 nmol/L except for 5(S)-HpETE (LLOQ=143 nmol/L) and AA (LLOQ=233 nmol/L)
Typical assay range in plasma is 1-500 nmol/L for prostanoids and hydroxylated fatty acid metabolites
Coefficient of variation (CV) for intraday and interday precision, and accuracy at three concentrations was determined.
Recoveries were found between 70-120% depending on the metabolite.

The method described was applied to plasma, sera, liver, brain and prostate homogenates. Free fatty acid metabolites could be identified and quantified without need for any derivatization or evaporation steps. FIGS. 3a, b, c, and d show the TICs of oxidized fatty acid metabolites extracted from human serum, brain homogenate, liver homogenate (murine) and prostate tissue (human), respectively.

Test cases of disease states show an increase of free fatty acids, prostaglandins and hydroxylated species in conjunction with pulmonary inflammations, prostate cancer and cardiovascular disease. As an example the method was applied to a nephrotoxicity model since the oxidative modification of low density lipoproteins (LDL) including oxidation of arachidonic acid is evidence of oxidative stress and inflammatory processes in kidney degeneration:

Plasma samples obtained from 4 groups of rats receiving different dosages of puromycin were analyzed. Increased cyclooxygenase and lipoxygenase activity was observed as shown in FIG. 4.

Detection of Eicosanoids Together with Oxidative Stress Parameters in Parallel:

Detection of compounds from different metabolite classes in parallel was performed in two different ways, depending on the chromatographic characteristics of the metabolite classes:
1) Same extraction from sample and detection in one LC-MS/MS run
2) Same extraction from sample and detection in different LC-MS/MS runs

Three examples will be described.

ad 1) Methionine (Met), methioninesulfoxide (Met(O))

FIG. 5 shows a chromatographic separation of Met, Met(O), D₃-Met and 6 prostaglandins (PGD₂, PGE₂, PGF_{2α}, 8-iso PGF_{2α}, 6-keto PGF_{1α} and TXB₂). D₃-Met was used as internal standard for Met and Met(O). Standard solutions of Met, Met(O), D₃-Met (c=50 μM for all) and PGs (c=1 μM for all) were extracted in parallel as described above and injected into the LC-MS/MS system. Detection of Met, Met(O) and D3-Met was achieved in positive ion mode, after t=3 min data acquisition was switched to negative mode to detect the prostanoids.

### α-Ketoglutarate, Succinate

FIG. 6 shows a chromatographic separation of α-ketoglutarate, succinate and 6 PGs. Concentrations of α-ketoglutarate and succinate were 100 μM, concentrations of PGs c=1 μM. Analytes were extracted in parallel as described above and injected into the LC-MS/MS system. Detection was performed in negative ion mode.

### ad 2) 4-Hydroxynonenal (4-HNE)

FIG. 7 shows a chromatographic separation of 4-HNE and the internal standard 4-HNE-d₃ with concentrations of 16 μM. 4-HNE, 4-HNE-d₃ and 6 PGs were extracted as described above and injected into the LC-MS/MS system. Detection was performed in positive ion mode. Due to the chromatographic characteristics of 4-HNE (elution at high organic content—90% MeOH—in mobile phase at t=1.1 min), extraction of 4-HNE and eicosanoids is performed in parallel, however detection has to be performed in two different LC-MS/MS runs.

### BRIEF DESCRIPTION OF FIGURES

FIG. 1. Scheme of extraction process: application of sample on insert in microtiter plate, extraction with aqueous methanol (MeOH), centrifugation, analysis by LC-MS/MS without derivatization and solid phase extraction.

FIGS. 2a and 2b. Chromatographic separation of an external standard mixture of free fatty acids, prostanoids, isoprostanes and LOX- and Cytochrom P 450-derived fatty acid metabolites.

FIGS. 3a, 3b, 3c and 3d. Detection of various eicosanoids and fatty acid derivatives in a selection of biological samples (as indicated).

FIGS. 4a, 4b, 4c and 4d. Effect of different puromycin dosages in rats. The concentrations of 4 different eicosanoids (as indicated) in rat plasma samples have been determined and normalized.

FIG. 5. Standard separation of Met, D₃-Met, Met(O) (c=50 μM) and 6 PGs (c=1 μM), extracted with 85% MeOH, in a single LC-MS/MS run. Column: Zorbax Eclipse XDB C18, 100×3 mm, 3.5 μm. Mobile phases: A=H2O, 0.2% formic acid, B=ACN, 0.05% formic acid; gradient elution, flow=500 μL/min, injection volume=20 μL. Positive ionization mode t=0-3.0 min, negative ionization mode t=3.0-9 min.

FIG. 6. Standard separation of α-ketoglutarate, succinate (c=100 μM) and 6 Prostaglandins (c=1 μM), extracted with 85% MeOH, in one single LC-MS/MS run. Column: Zorbax Eclipse XDB C18, 100×3 mm, 3.5 μm. Mobile phases: A=95/5H2O/ACN, 15 mM NH4Ac, pH=5.2; B=95/5 ACN/H2O, 15 mM NH4Ac, pH=5.2; gradient elution, flow=500 μL/min, injection volume=20 μL, negative acquisition.

FIG. 7. Standard separation of 4-HNE and 4-HNE-d₃ (c=16 μM), extracted with 85% MeOH. Column: Zorbax Eclipse XDB C18, 100×3 mm, 3.5 μm. Mobile phases: A=H₂O, 0.05% formic acid; B=MeOH, 0.05% formic acid; isocratic elution, 10% A; flow=500 μL/min, injection volume=20 μL, positive ionization mode.

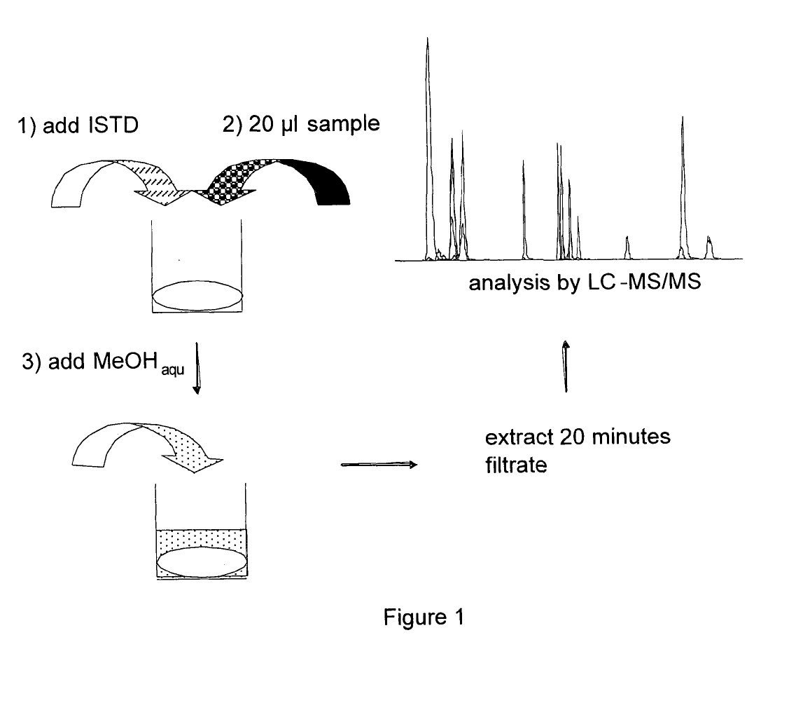

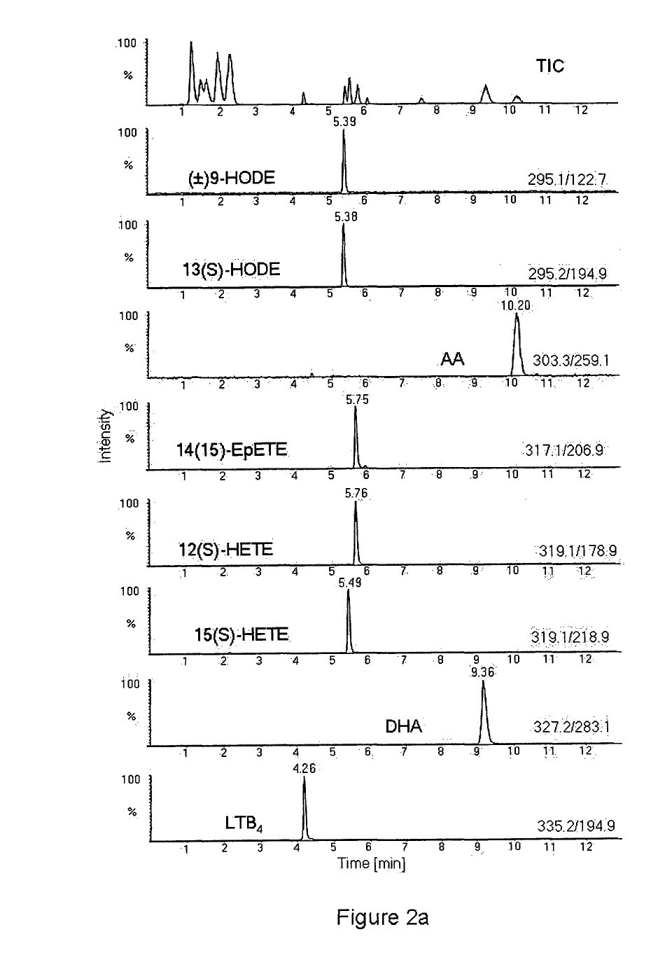

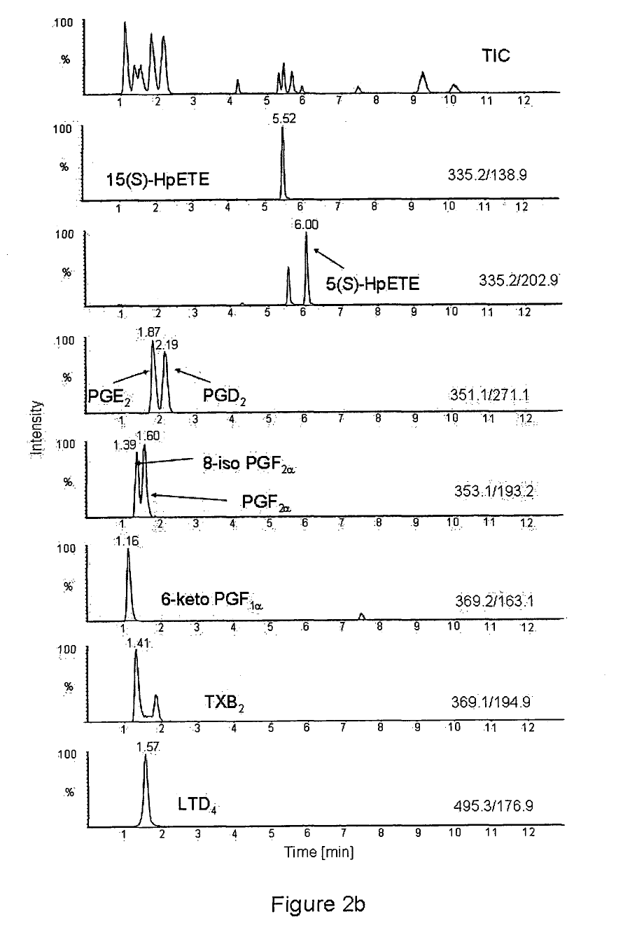

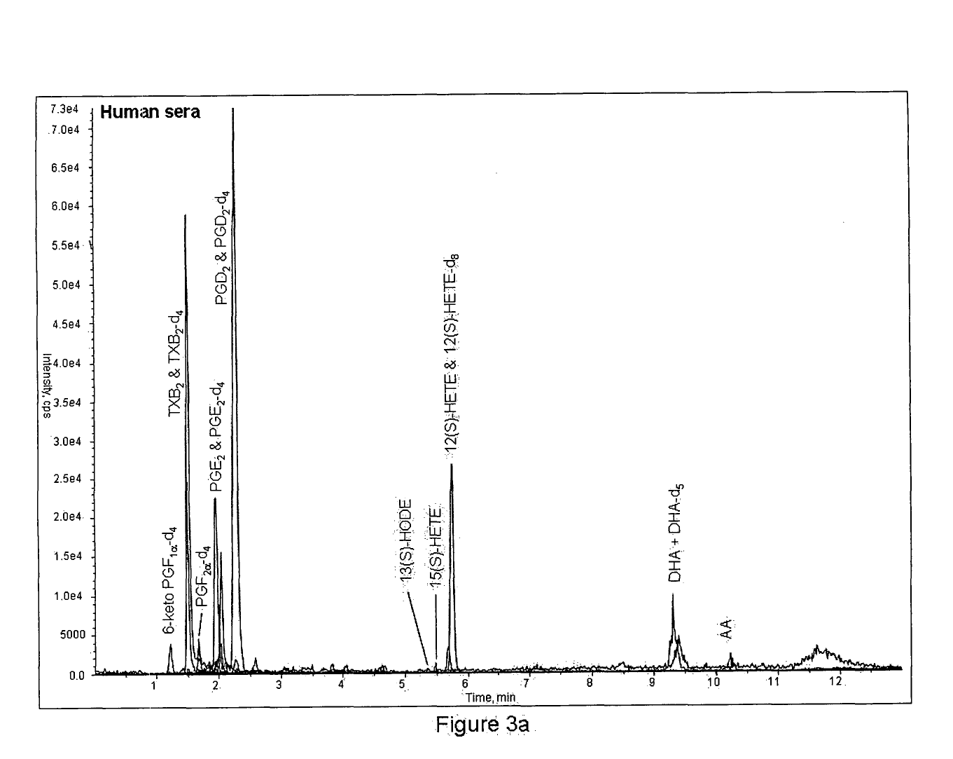

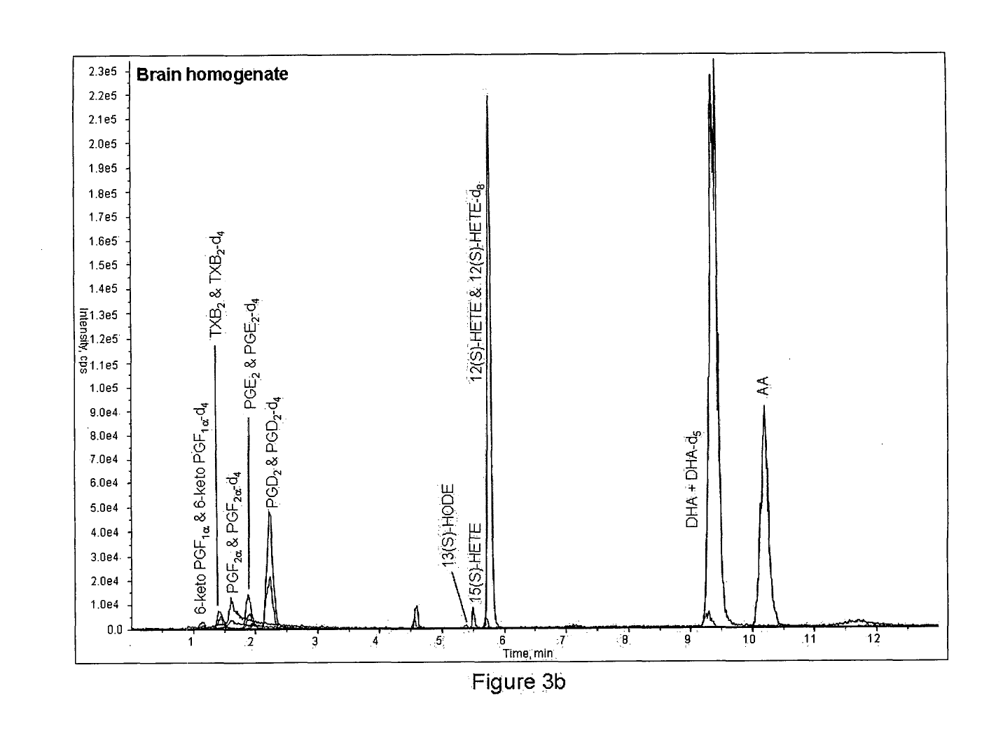

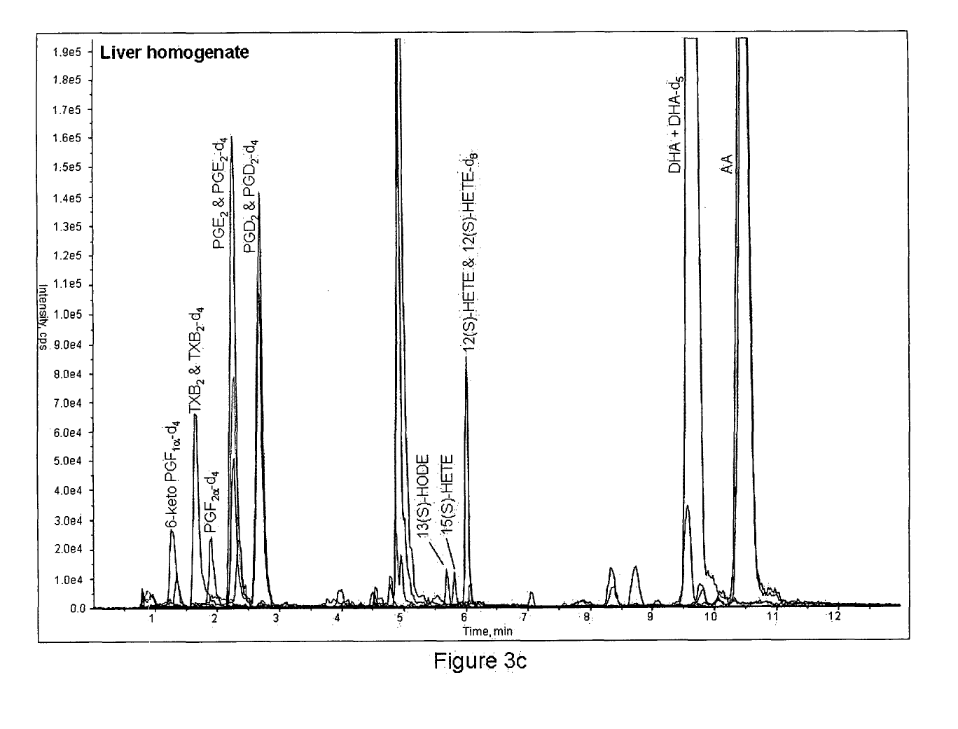

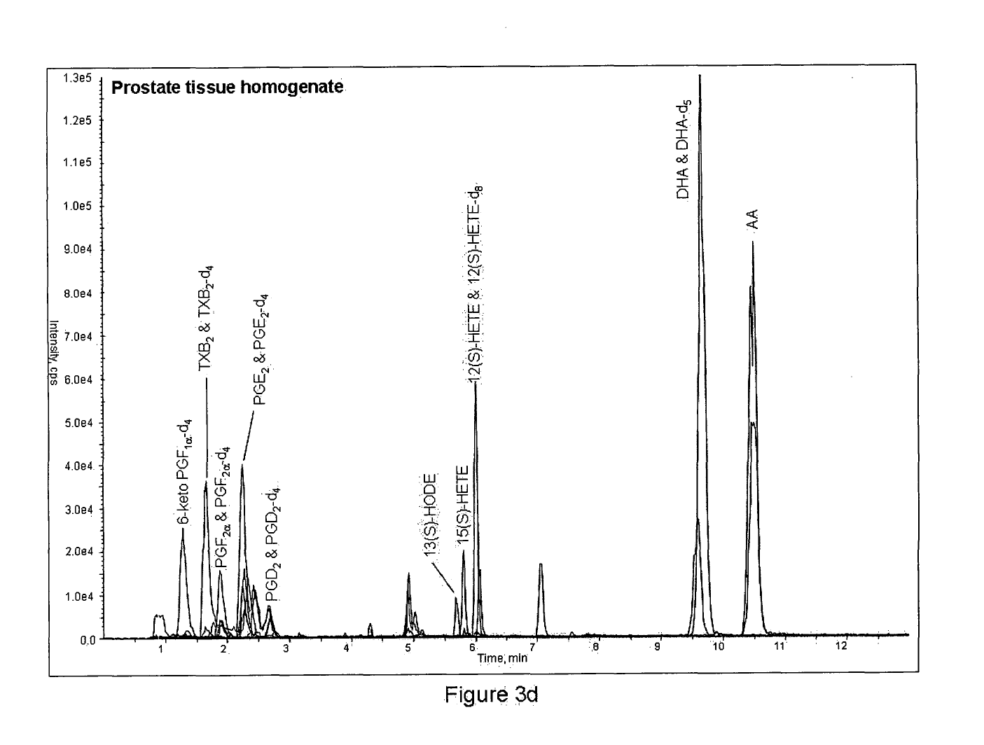

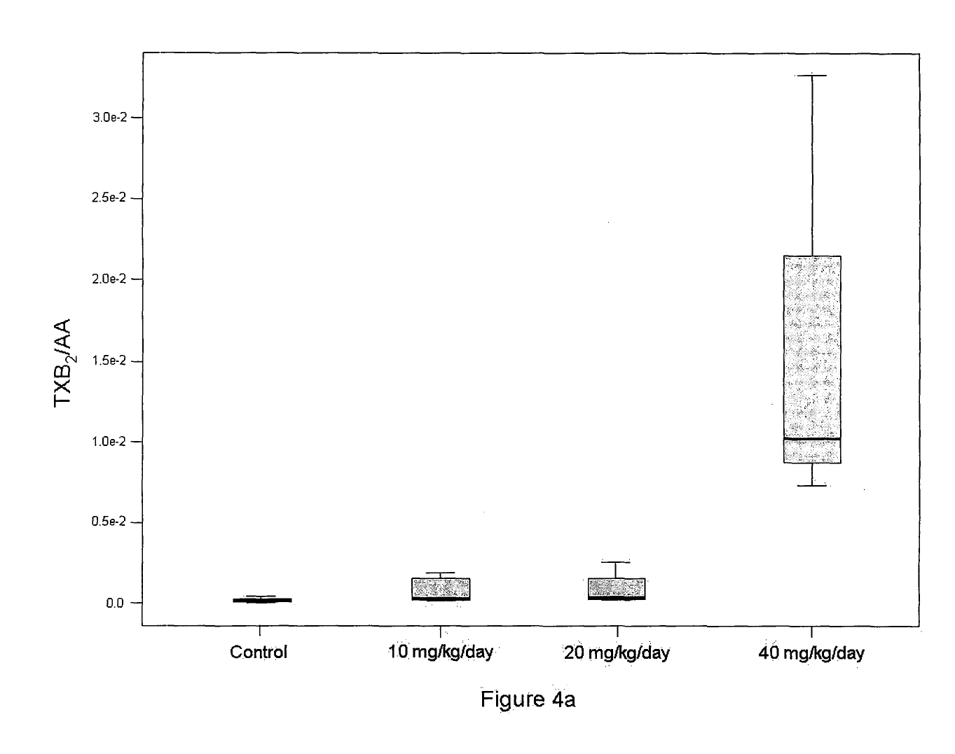

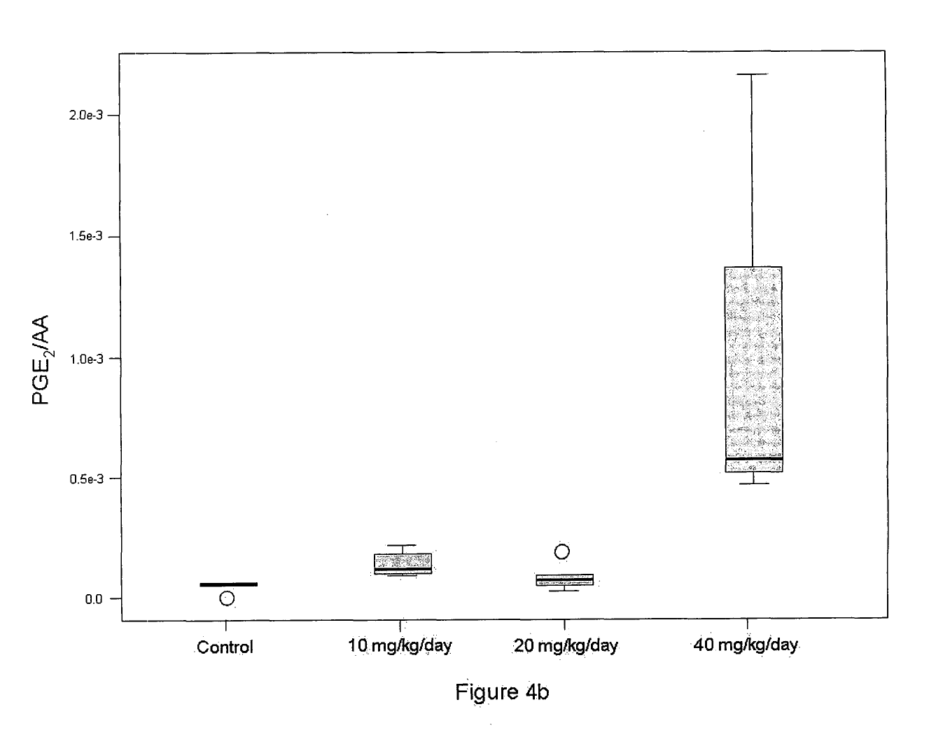

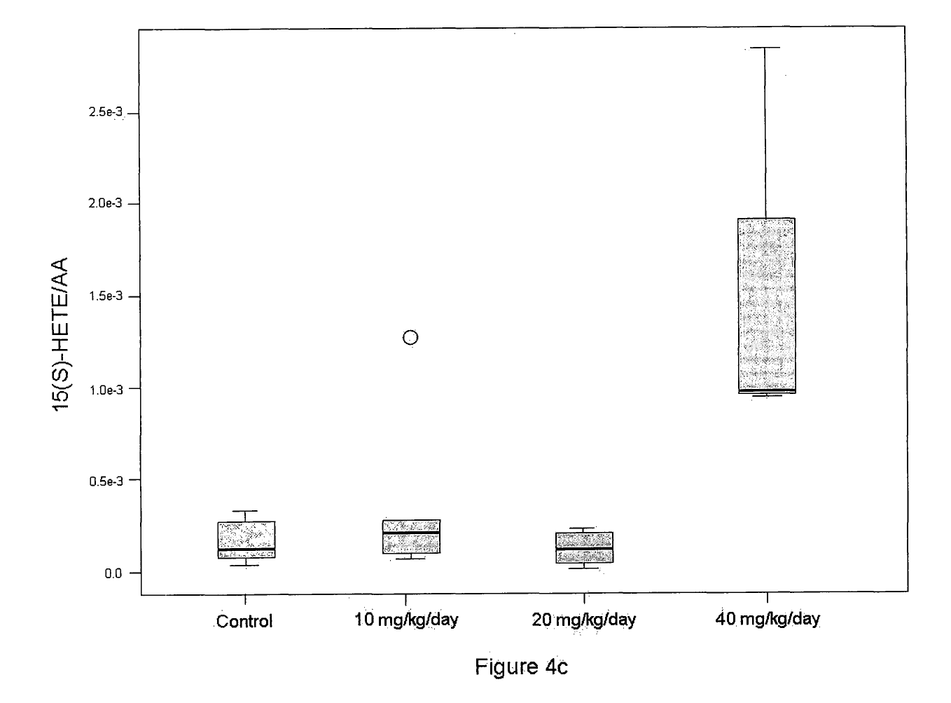

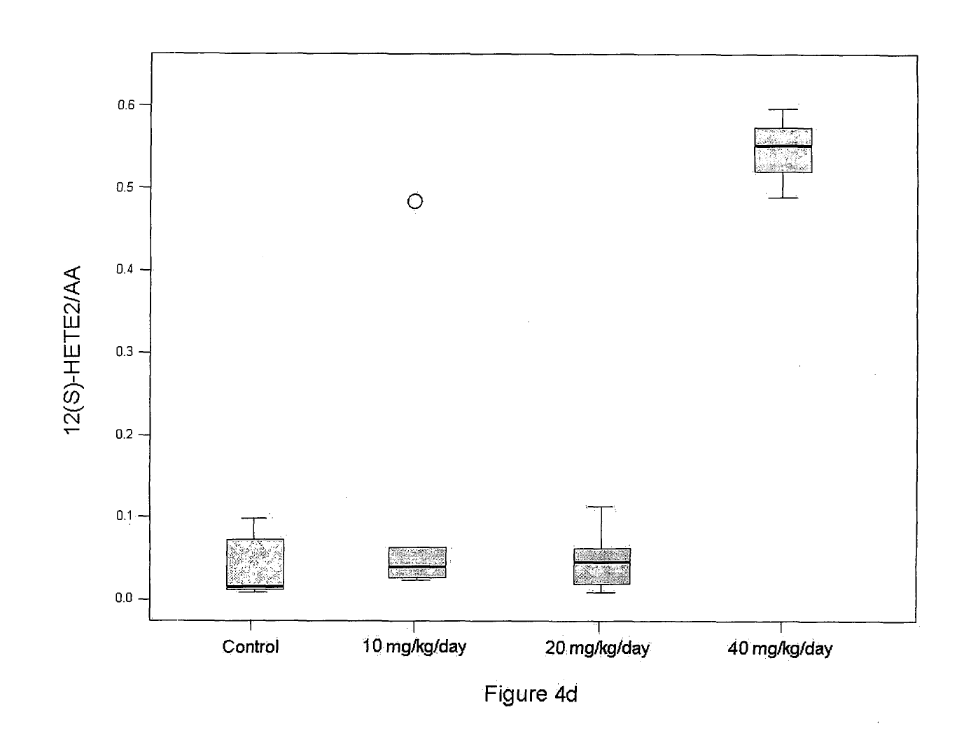

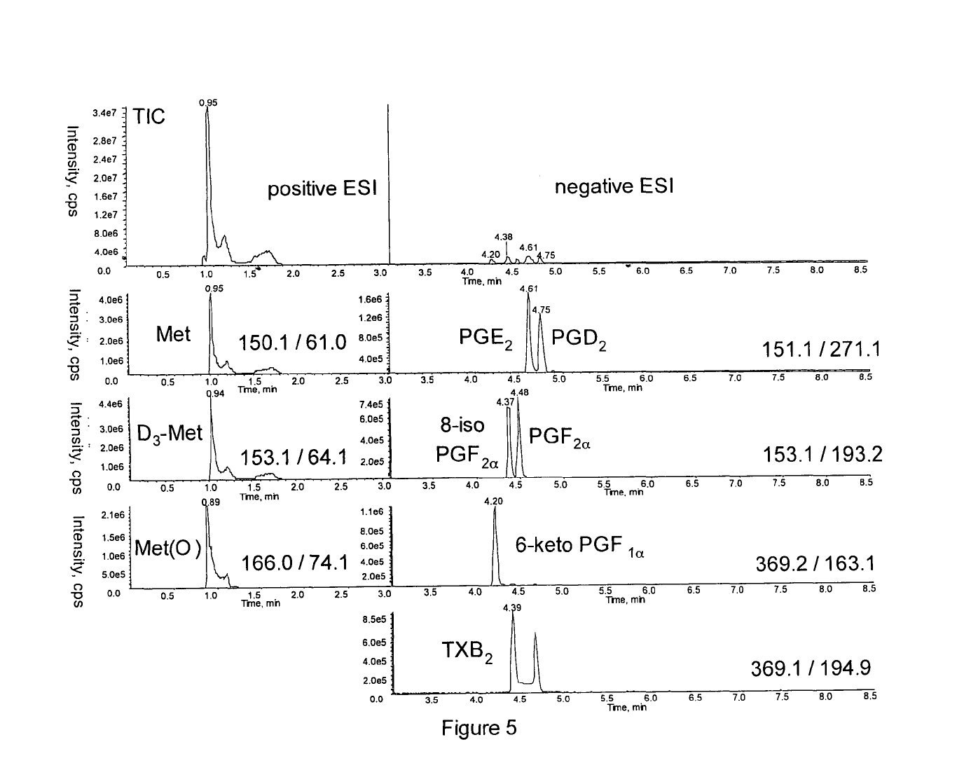

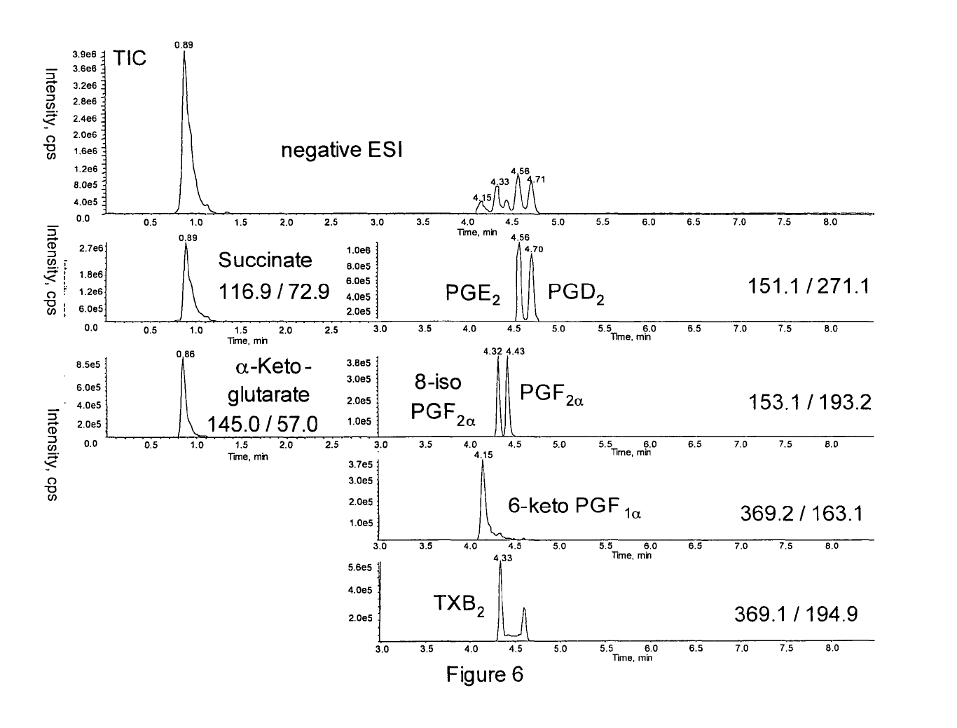

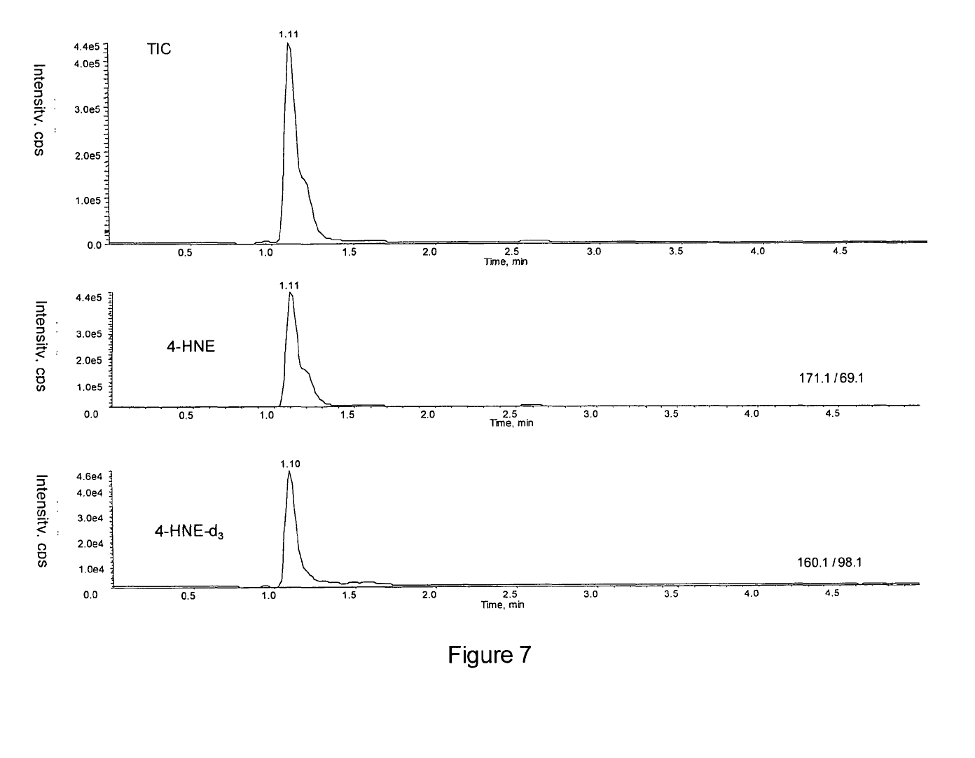

### INDUSTRIAL APPLICABILITY

The present invention provides for an improved method for determining the systemic metabolic status in relation to inflammation and oxidative stress in a biological sample. This method is highly sensitive and allows for the detection of only slight changes in the systemic metabolic status. The method comprises the detection and quantification of one or more derivatives of arachidonic acid (eicosanoids), of linoleic acid and/or of docosahexaenoic acid (docosanoids). Preferably one or more oxidative stress parameters are detected and quantified in parallel in order to further increase the sensitivity of the method and the quality of the results. The method is further improved by additionally detecting and quantifying one or more analytes from other metabolite classes in parallel. Thus, in a particularly preferred embodiment of the present invention three groups of compounds are detected and quantified in parallel which highly improves the sensitivity and reliability of the method (assay) with respect to the systemic metabolic status of a biological source in relation to inflammation and oxidative stress.

Thus, the method described in the present invention allows the parallel determination of metabolites related to inflammation and oxidative stress in a biological sample. This is necessary to enable a comprehensive evaluation of the systemic metabolic status, particularly for the purpose of differential diagnostics. A further advantage is based on the fact that the procedure has both high sensitivity and selectivity, and needs a very low sample volume, i.e. approximately 20 μL.

Potential therapeutic targets to be screened according to the method of the invention include a broad spectrum of human medical conditions such as various types of cancers, diabetes, obstructive lung disease, inflammatory bowel disease, cardiac ischemia, glomerulonephritis, macular degeneration and various neurodegenerative disorders. The method of the invention is also useful in detecting the gradual change of the systemic metabolic status, e.g. due to therapeutic effects. Thus, the method and the kit for carrying out the method are highly efficient tools in numerous medical fields, both in diagnosis and therapy.

1. A method for determining a systemic metabolic status in relation to inflammation and/or oxidative stress in a biological sample, which comprises detection and quantification of one or more compounds or derivatives thereof selected from the group consisting of arachidonic acid (eicosanoids), linoleic acid, docosahexaenoic acid, and combinations thereof.

2. The method according to claim 1, further comprising detection and quantification of an oxidative stress parameter selected from the group consisting of products of lipid oxidation and/or peroxidation, tyrosine derivatives including NO₂-, Br-, Cl-tyrosine, methionine sulfoxide, ketone bodies, 8-oxo-guanidine and 8-OH guanosine, biopterins, pro-vitamins, vitamins, antioxidants, glutathione, ophthalmate, oxidised cholesterols and sterols, and combinations thereof.

3. The method according to claim 1, further comprising detection and quantification of an analyte from other metabolite classes selected from the group consisting of α-ketoglutarate, succinate, CoQ₁₀, methionine, sphingolipids including ceramide-1-phosphate, sphingosine-1-phosphate, sphingomyelins and hydroxylated sphingomyelins, and combinations thereof.

4. The method according to claim 1, wherein the detection is carried out by measuring one or more metabolite concentrations.

5. The method according to claim 1, wherein the detection is carried out in a sample having a volume within a range of from 1 μl to 1 ml.

6. The method according to claim 1, wherein the detection is carried out without any usual sample preparation procedures such as derivatization of the metabolites and/or liquid-liquid or solid phase extraction of the metabolites.

7. The method according to claim 1, wherein the biological sample is obtained from a mammal, including from a mouse, a rat, a guinea pig, a dog, a mini-pig, a primate or a human.

8. The method according to claim 1, wherein the detection is based on a quantitative analytical method, including chromatography, spectroscopy, and mass spectrometry.

9. (canceled)

10. The method according to claim 1, wherein the derivatives of arachidonic acid are selected from the group consisting of arachidonic acid and its metabolites, including cyclooxygenase-, lipoxygenase- and cytochrome P450-derived prostanoids, hydroxy-, hydroperoxy- and epoxylated acids and non-enzymatic peroxidation products including isoprostanes.

11. The method according to claim 1, wherein the derivatives of linoleic acid are selected from the group consisting of linoleic acid and its metabolites, including lipoxygenase- and cytochrome P450-derived oxidation products, and non-enzymatic peroxidation products.

12. The method according to claim 1, wherein the derivatives of docosahexaenoic acid are selected from the group consisting of docosahexaenoic acid and its metabolites, including lipoxygenase- and cytochrome P450-derived docosanoids and non-enzymatic peroxidation products including isoprostanes.

13. The method according to claim 1, wherein the systemic metabolic status is indicative for various cancer types, inflammatory diseases including chronic airway inflammation or atherosclerosis, and metabolic disorders including diabetes.

14. A kit comprising a device which device comprises one or more wells and one or more inserts impregnated with at least one internal standard, wherein the kit is adapted for carrying out the method according to claim 1.

15. A biomarker for determining a systemic metabolic status in relation to inflammation and/or oxidative stress in a biological sample, which comprises one or more compounds or derivatives thereof selected from the group consisting of arachidonic acid (eicosanoids), linoleic acid, docosahexaenoic acid and combinations thereof.

16. The biomarker according to claim 15, further comprising detection and quantification of an oxidative stress parameters-Ewe selected from the group consisting of products of lipid oxidation and/or peroxidation, tyrosine derivatives including NO₂-, Br-, Cl-tyrosine, methionine sulfoxide, ketone bodies, 8-oxo-guanidine and 8-OH guanosine, biopterins, pro-vitamins, vitamins, antioxidants, glutathione, ophthalmate, oxidised cholesterols and sterols, and combinations thereof.

17. The biomarker according to claim 15, further comprising detection and quantification of an analytes from other metabolite classes selected from the group consisting of α-ketoglutarate, succinate, CoQ₁₀, methionine, sphingolipids, including ceramide-1-phosphate, sphingosine-1-phosphate, sphingomyelins and hydroxylated sphingomyelins, and combinations thereof.

18. The biomarker according to claim 15, wherein the derivatives of arachidonic acid are selected from the group consisting of arachidonic acid and its metabolites, including cyclooxygenase-, lipoxygenase- and cytochrome P450-derived prostanoids, hydroxy-, hydroperoxy- and epoxylated acids and non-enzymatic peroxidation products, including isoprostanes.

19. The biomarker according to claim 15, wherein the derivatives of linoleic acid are selected from the group consisting of linoleic acid and its metabolites, including lipoxygenase- and cytochrome P450-derived oxidation products, and non-enzymatic peroxidation pro ducts.

20. The method according to claim 15, wherein the derivatives of docosahexaenoic acid are selected from the group consisting of docosahexaenoic acid and its metabolites, including lipoxygenase- and cytochrome P450-derived docosanoids and non-enzymatic peroxidation products including isoprostanes.

21. The biomarker according to claim 15, wherein the systemic metabolic status is indicative for various cancer types, inflammatory diseases including chronic airway inflammation or atherosclerosis, and metabolic disorders including diabetes.
